(19) United States
(12) Patent Application Publication
(10) Pub. No.: US 2020/0100993 A1
(43) Pub. Date: Apr. 2, 2020

(54) USE OF REVITALIZING COSMETIC COMPOSITION AND NON-THERAPEUTIC METHODS

(71) Applicant: LUXBIOTECH FARMACEUTICA LTDA, Jaguariuna (BR)

(72) Inventors: Silvana Masiero, Hortolandia (BR); Luiz Felipe de Oliveira Stehling, Hortolandia (BR); Claudia Marisa Antunes Marcal, Campinas (BR); Jean-Alexis Grimaud, Paris (FR); Beatrice Muscatelli-Groux, Paris (FR)

(21) Appl. No.: 16/442,095

(22) Filed: Jun. 14, 2019

(30) Foreign Application Priority Data
Jun. 14, 2018 (BR) 10-2018 012093 0

(51) Int. Cl. A61K 8/19; A61Q 19/08; A61N 5/06

(52) U.S. Cl. CPC A61K 8/19; A61Q 19/08; A61N 5/062; A61N 2005/0662; A61K 2800/81; A61N 2005/0652; A61N 2005/0663; A61N 5/0616

(57) ABSTRACT

The present invention relates to the use of revitalizing cosmetic compositions in association with a light emitting diode (LED) as skin cell stimulators and a related non-therapeutic method for the skin treatment of aging and for repair and recovery of cutaneous tissue.

### FIELD OF THE INVENTION

This invention relates to the use of revitalizing cosmetic compositions in association with a light emitting diode (LED) as skin cell stimulators and a related non-therapeutic method for the skin treatment of aging and for repair and recovery of cutaneous tissue.

### BACKGROUND OF THE INVENTION

The exposure of the skin to a variety of agents causes its extrinsic aging. These agents can be any of the following causes: pollution, genetic dispositions, solar radiation and UV exposure, among others. In addition to external factors, internal agents are responsible for intrinsic aging. Skin aging can be defined as a fall and/or decay of the rate of the metabolism efficiency due to insufficient production of substances and cells to promote tissue regeneration. Moreover, due to the loss of elasticity and firmness and consequent fragility, the skin aging can be characterized as being intrinsic when there is the identification of a superficial wrinkling, as well as thinning and sagging of the skin, or being extrinsic, when the presence of deep wrinkles and pockets, dryness, and pigmentation irregularities is noted (Emerit, I. “Free Radicals and Aging of the Skin”. Birkhäuser Basel 1992; 62:328-341).

In order to solve the problem of skin aging, various non-therapeutic techniques and forms of non-therapeutic treatment are suggested in the literature and by another important segment, the cosmetic industry, which promotes treatments using active ingredients incorporated into creams, cosmetic compositions, gels, elixirs among others.

In addition, it is considered that the associated use of different treatment methods, such as cosmetics and chemicals, has a trend to provide a significant improvement in the skin, contributing to its treatment and rejuvenation.

There are several prior art documents relating to methods and compositions used to improve and/or treat skin aging. Among them, reference may be made to ZA1998/00404, which relates to a cosmetic skin treatment composition comprising from 0.1% to 75% of at least one cellular therapeutic compound, substance or composition, that is selected from hyaluronic acid or a pharmaceutically acceptable salt thereof, a keratin binding complex, a collagen amino acid, a composition including sericin and glycoprotein, among others, in addition to a cosmetically acceptable carrier or diluent. Further it discloses a substance or composition for use in a method of treating the human skin, wherein in said method there is the application to the skin of an effective amount of the substance or composition with subsequent incidence of laser irradiation to the subject's skin who needs treatment.

The document US20130115180 relates to compositions for administering electromagnetic radiation (EMR) for therapeutic or cosmetic purposes and methods, wherein said compositions comprise a carrier lotion of active cosmetic ingredients which is suitable for application to human skin; a first and second compositions suitable for application to the human skin, wherein the first preferably blocks the EMR below a certain predetermined wavelength range and the second preferably blocks the EMR above another predetermined wavelength range; and the source of light used may be artificial or ultraviolet light.

Thus, it is still desirable to find topically applicable compositions for skin treatment which, when exposed to a particular type of radiation, especially radiation from light emitting diode (LED), are able to show significant improvement results in relation to the issue of skin aging and in the recovery of cutaneous tissue, more specifically that they present cellular stimulation, and consequently, reduction of the aging signs and also, the repair of cutaneous tissue.

### SUMMARY OF THE INVENTION

As previously mentioned, this invention relates to the use of revitalizing cosmetic compositions which by photobiomodulation (the term representing cell stimulation from a light-emitting diode) on certain LED wavelengths, amplify the synthesis of certain essential proteins and expressed in fibroblast culture, such as collagen I, collagen III, fibronectin, laminin, α-SMA.

A preferred embodiment of the invention refers to the use of the revitalizing cosmetic compositions, particularly elixirs comprising growth factors, platinum, diamond and gold particles-conjugates peptides, pearl extract and caviar hydrolysate in combination with LED for optimization of the synthesis of cellular proteins selected from the group consisting of collagen I, collagen III, fibronectin, laminin and α-SMA.

In another preferred embodiment of the invention, there is disclosed a non-therapeutic method for the skin aging treatment, said method comprising combining revitalizing cosmetic compositions, in particular elixirs comprising growth factors, platinum, diamond and gold particles-conjugated peptides, pearl extract and caviar hydrolysate, with LEDs at certain wavelengths.

In yet another embodiment of the invention, there is disclosed a non-therapeutic method for repairing and recovering cutaneous tissue, said method also comprising the association of revitalizing cosmetic compositions, in particular elixirs comprising growth factors, platinum, diamond and gold particles-conjugated peptides, pearl extract and caviar hydrolysate, with LEDs at certain wavelengths.

Unexpectedly, the inventors of this invention have noted a synergistic potentiating action by associating the revitalizing cosmetic compositions with the wavelength lighting determined in the visible/LED light spectrum, continuously emitted over a given time. The subject matter of the invention is therefore a novel non-therapeutic method of skin aging treatment. It is a method that combines “in vitro” light (photobiomodulation) and revitalizing cosmetic compositions (bioinduction) for repair and recovery of cutaneous tissue, in which a set of induced cellular proteins is measured against the applied lighting—expressing a cellular metabolic state that determines new regeneration capacities.

These features of the invention will be described in more detail in the detailed description of the invention.

### BRIEF DESCRIPTION OF THE FIGURES

FIG. 1 shows qualitatively the increase in laminin expression due to the synergistic association of the use of 1% of the gold elixir and exposure to the various LED wavelengths.

FIG. 2 shows qualitatively the increase in collagen I and III expression due to the synergistic association of the use of 1% of the diamond elixir and exposure to the various LED wavelengths.

FIG. 3 shows qualitatively the increase of α-SMA expression as a result of the synergistic association of the use of 1% of the diamond elixir and exposure to the various LED wavelengths.

FIG. 4 shows qualitatively the increase in fibronectin expression due to the synergistic association of the use of 1% of the gold elixir or diamond elixir and exposure to the various LED wavelengths.

FIGS. 5 to 10 show graphs where the structural proteins and extracellular matrix elements are quantified.

FIGS. 11 to 12 show the migration tests performed for fibroblasts.

### DETAILED DESCRIPTION OF THE INVENTION

It is presented in this description the use of revitalizing cosmetic compositions in association to LED to optimize the extracellular matrix protein synthesis, and further the non-therapeutic method for skin aging treatment and repairing and recovering cutaneous tissue, said methods comprising the association of revitalizing cosmetic compositions, in particular elixirs comprising growth factors, platinum, diamond and gold particles-conjugated peptides, pearl extract and caviar hydrolysate, with LEDs at certain wavelengths.

By associating the revitalizing cosmetic compositions with the light emitting diode (LED), and evaluating the synthesis of structural proteins, the inventors surprisingly observed a potentiating synergistic effect of the association of the cosmetic/light compositions on the culture of fibroblasts compared to cultures of cells maintained in the absence of illumination.

The revitalizing cosmetic compositions of this invention are formulated as follows:

Pearl and Caviar Elixir: formulated to hydrate, inhibit tyrosinase and revitalize the skin. The high content of proteins and vitamins from pearl and caviar extracts activates new skin cells gradually reducing the formation of wrinkles, in addition to hydrating and helping to prevent loss of skin elasticity, promoting a more uniform texture.

Colloidal Gold Elixir: elixir specially formulated to remineralize the skin layers, with antioxidant and stimulus action to the synthesis of structural proteins of the skin, such as collagen (collagen I and III) and elastin. It stimulates healing, fibroblasts, filling and conduction of bioelectricity. The peptide conjugated with modified gold nanomaterial hydrates, revitalizes and unifies the texture of the cutaneous tissue, conferring to it, softness and luminosity.

Platinum Elixir: elixir specially formulated to restore the skin barrier by maintaining epithelial thickness through hyaluronic acid, GAG structural proteins and Langerhans cells, reducing the dermal-epidermal cell matrix degradation and acting on healing. The enzyme peptide conjugated with platinum nanomaterial enhances the production of structural proteins, hydrates and protects the skin against environmental stress, conferring vitality and protection.

Diamond Elixir—elixir specially formulated to reshape and rebuild skin structures through collagen VII, laminin-5, fibronectin, reducing wrinkles and expression lines, improving healing and filling. The high concentration of available peptide with diamond nanomaterial hydrates and helps preventing loss of skin elasticity, giving young and healthy appearance with renewed texture.

The structural proteins expressed in the extracellular matrix are selected from the group consisting of: collagen I, collagen III, fibronectin, laminin and α-SMA

The non-therapeutic method for the skin aging treatment of this invention therefore comprises the following steps:
a) applying an acceptable amount of at least one revitalizing cosmetic composition onto the cutaneous tissue;
b) subjecting the revitalizing cosmetic composition applied in (a) light having wavelengths between 410 nm and 780 nm;
c) maintaining the exposure of the revitalizing cosmetic composition to light as described in (b) for a time period ranging from at least 15 seconds to 2 minutes.

The non-therapeutic method for repairing and recovering cutaneous tissue comprises the following steps:
a) applying an acceptable amount of at least one revitalizing cosmetic composition onto the injured or discontinued cutaneous tissue; 0.35 to 70 ml
b) subjecting the revitalizing cosmetic composition applied in (a) light having wavelengths between 410 nm and 780 nm;
c) maintaining the exposure of the revitalizing cosmetic composition as described in (b) for a time period ranging from 5 hours to 40 hours, more preferably, between 20 hours to 40 hours.

In a preferred embodiment of the invention the amount of the revitalizing cosmetic composition ranges from 0.35 ml to 15 ml, more preferably the amount of the revitalizing cosmetic composition to be applied to the skin tissue is from 0.35 ml to 1.50 ml.

The inventors when measuring cutaneous markers/cellular proteins from a control sample where only the revitalizing cosmetic composition was applied and compared with samples that received the revitalizing cosmetic composition and were also subjected to LED light with wavelengths ranging from 410 nm and 780 nm, surprisingly obtained the results as shown in Tables 1 to 5 as follows:

TABLE 1

| | Collagen 1 | | | | | | | | | | |
|---|---|---|---|---|---|---|---|---|---|---|---|
| ELIXIR 1% | Sem LUZ | 410 nm | 440 nm | 470 nm | 520 nm | 590 nm | 630 nm | 660 nm | 690 nm | 780 nm | 470-850 nm |
| CTRL | * | | | | | | | | | | |
| Pearl & Cavlar | * | * | * | ** | * | ** | * | * | ** | * | * |
| Gold | * | ** | ** | ** | * | ** | ** | * | ** | * | |
| Platin | * | * | * | ** | * | *** | ** | * | * | * | * |
| Diamond | ** | ** | ** | ** | ** | *** | *** | *** | * | ** | * |

TABLE 2

| | Collagen III | | | | | | | | | | |
|---|---|---|---|---|---|---|---|---|---|---|---|
| ELIXIR 1% | Sem LUZ | 410 nm | 440 nm | 470 nm | 520 nm | 590 nm | 630 nm | 660 nm | 690 nm | 780 nm | 470-850 nm |
| CTRL | | | | | | | | | | | |
| Pearl & Cavlar | * | * | * | * | * | * | * | * | * | * | * |
| Gold | * | * | * | * | * | * | * | * | * | * | * |
| Platin | * | * | * | * | * | * | * | * | * | * | * |
| Diamond | * | ** | ** | ** | ** | *** | *** | ** | ** | ** | *** |

TABLE 3

| | fibronectin | | | | | | | | | | |
|---|---|---|---|---|---|---|---|---|---|---|---|
| ELIXIR 1% | Sem LUZ | 410 nm | 440 nm | 470 nm | 520 nm | 590 nm | 630 nm | 660 nm | 690 nm | 780 nm | 470-850 nm |
| CTRL | * | | | | | | | | | | |
| Pearl & Cavlar | * | * | * | * | * | * | ** | * | ** | * | * |
| Gold | ** | ** | ** | *** | *** | *** | *** | ** | * | * | ** |
| Platin | * | * | * | * | * | * | * | ** | ** | ** | * |
| Diamond | ** | ** | *** | *** | *** | *** | *** | *** | ** | ** | * |

TABLE 4

| | αSMA (myofibroblasts) | | | | | | | | | | |
|---|---|---|---|---|---|---|---|---|---|---|---|
| ELIXIR 0.15% | Sem LUZ | 410 nm | 440 nm | 470 nm | 520 nm | 590 nm | 630 nm | 660 nm | 690 nm | 780 nm | 470-850 nm |
| CTRL | * | | | | | | | | | | |
| Pearl & Cavlar | * | * | ** | ** | ** | * | ** | ** | * | / | / |
| Gold | * | * | ** | * | * | ** | ** | ** | ** | / | / |
| Platin | ** | ** | ** | ** | ** | *** | ** | ** | ** | *** | / |
| Diamond | * | ** | ** | ** | ** | *** | ** | * | * | / | / |

TABLE 5

| | Laminin | | | | | | | | | | |
|---|---|---|---|---|---|---|---|---|---|---|---|
| ELIXIR 1% | Sem LUZ | 410 nm | 440 nm | 470 nm | 520 nm | 590 nm | 630 nm | 660 nm | 690 nm | 780 nm | 470-850 nm |
| CTRL | * | | | | | | | | | | |
| Pearl & Cavlar | * | * | * | * | * | * | * | * | * | * | / |
| Gold | * | * | ** | *** | *** | ** | * | ** | ** | ** | ** |
| Platin | * | * | * | * | * | * | * | * | * | * | / |
| Diamond | * | * | * | * | * | * | * | * | * | * | / |

* A red dot corresponding to the control or base level. Two red dots indicate that the skin marker production was moderately larger than that of the control. Three red dots indicate that the production of the skin marker was significantly greater than that of the control.

According to this invention, the terms “revitalizing cosmetic compositions” and “elixirs” are used interchangeably and refer to compositions for treating the aging signs.

Furthermore, the terms “skin markers” and “certain essential proteins” are used interchangeably and refer to proteins synthesized by fibroblasts, and make up the extracellular matrix.

The revitalizing cosmetic compositions or elixirs are formulated with excipients, humectants, preservatives, opacifier, thickener, skin protectant, conditioner, colorant, emollient, chelating, emulsifier, pH adjuster, fragrance, growth factor, and further, vehicle options.

Cosmetically acceptable excipients include, without limitation, pH adjusting agents, conditioners, preservatives, colorants, emollients, emulsifiers, fragrances, thickeners, sequestering and carriers.

Examples of pH adjusting agents include, without limitation, acetic acid, citric acid, hydrochloric acid, lactic acid, sodium bicarbonate, ammonium carbonate, potassium hydroxide, sodium hydroxide, triethanolamine.

Examples of conditioners include, without limitation, copper acetylmethionate, magnesium acetylmethionate, manganese acetylmethionate, zinc acetylmethionate, methylsilanol hydroxyproline aspartate, carnosine and its derivatives, plant extracts such as olive leaf extract (Olea europaea), glutamylamidoethyl imidazole, vegetable oils such as cottonseed oil (Gossypium herbaceum), sunflower seed oil (Helianthus annuus), passion fruit seed oil (Passiflora edulis), grape seed oil (Vitis vinifera).

Examples of preservatives include, without limitation, benzoic acid, benzyl alcohol, sodium benzoate, cetylpyridinium chloride, benzalkonium chloride, benzethonium chloride, phenoxyethanol, imidazolinidyl urea, parabens and mixtures thereof.

Examples of colorants include, without limitation, CI10315 (yellow), CI12085 (red), CI15510 (orange), CI15800 (red), CI15880 (red), CI15985 (yellow), CI19140 (yellow), CI20170 (brown), CI42053 (green), CI42080 (blue), CI42090 (blue), CI42510 (violet), CI45100 (red), CI45370 (orange), CI59040 (green), CI60725 (violet), CI60730 (violet), CI75170 (white), caramel and mixtures thereof.

Examples of emollients include, without limitation, stearic acid, lactic acid, animal fats such as lanolin, vegetable oils such as cottonseed oil (Gossypium herbaceum), sunflower seed oil (Helianthus annuus), passion fruit seed oil (Passiflora edulis), grape seed oil (Vitis vinifera), propylheptyl caprylate, caprylyl glycol, coco caprylate/caprate, cyclomethicone, dimethicone, ethoxydiglycol, glycerin, lactose, cetyl palmitate, sorbitol, caprylic/capric acid triglyceride, urea.

Examples of emulsifiers include, without limitation, cetearyl alcohol, cetyl alcohol, stearyl alcohol and mixtures thereof, glyceryl stearate, ethoxylated fatty alcohols, sorbitan esters such as sorbitan oleate and sorbitan stearate, lecithin and polysorbates.

Examples of thickeners include, without limitation, waxes, such as beeswax, carnauba wax and lanolin wax and lanolin, polysaccharides, among which starch, gums such as arabic gum, guar gum, xanthan gum, tragacanth, agar, carrageenans and alginates, cellulose and its derivatives, such as microcrystalline cellulose, cellulose acetate, carboxymethylcellulose and hydroxyethylcellulose, glyceryl stearate, polyethylene glycol, polyvinylpyrrolidone, polyvinyl alcohol, carbopol, polyacrylic acid, silanes and derivatives, alkyl polyacrylates, alkyl polymethacrylates and mixtures thereof.

Examples of fragrances include, without limitation, natural, synthetic fragrances and mixtures thereof.

Examples of sequestrants include, without limitation, EDTA, disodium EDTA, tetrasodium EDTA, monobasic sodium phosphate and dibasic sodium phosphate and mixtures thereof.

Examples of carriers include, without limitation, water, esters in general, mineral oil and vegetable oils and mixtures thereof, when into an emulsified system.

As previously stated, control and exposure of cell culture with revitalizing cosmetic composition associated with LED stimulation at various wavelengths were compared and optimal wavelength ranges were obtained for the synergistic potentiation of elixir application with exposure to LED for each marker. A synergistic potentiating action was surprisingly detected in this associated use of revitalizing cosmetic compositions with LEDs, which provided an increase in the synthesis of cutaneous markers such as collagen I, collagen III, fibronectin, laminin and α-SMA.

### EXAMPLES

### Evaluations and Results

The methodology for measuring photobiostimulation was to compare the control and exposure of the cell culture with the product after stimulation of the LED at various wavelengths.

The analyzed wavelengths are in the range from 410 nm to 780 nm.

### Non-Therapeutic Method for the Cutaneous Aging Treatment:

Complementing the results of cell stimulation on fibroblasts from certain LED wavelengths, evaluation of cell migration on fibroblasts was determined using the associations between the parameters (concentration of cosmetic composition used, wavelength, time of exposure to LED, for example).

The fibroblast cell culture was submitted to LED and contact with the elixirs according to the best parameters of the qualitative evaluation (data from tables 1 to 5).

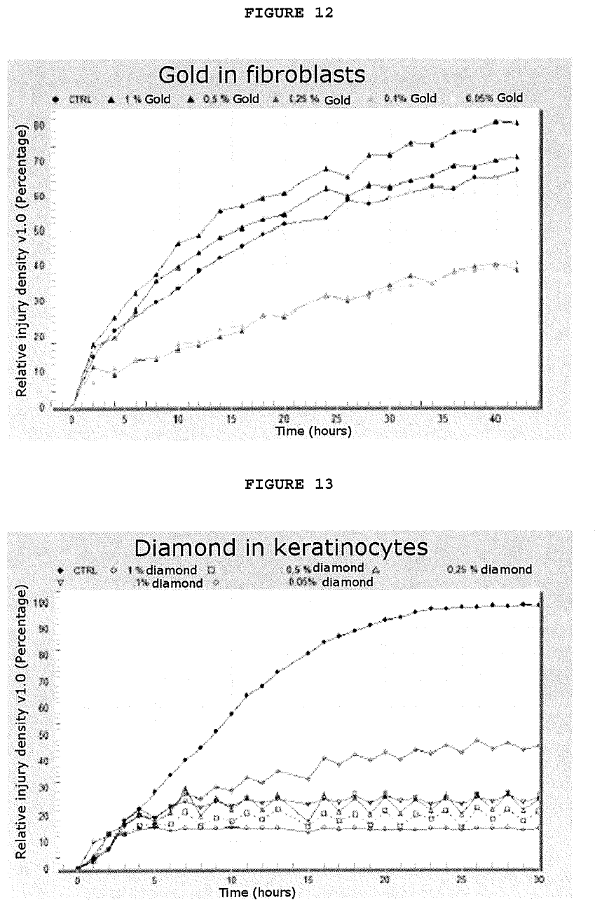

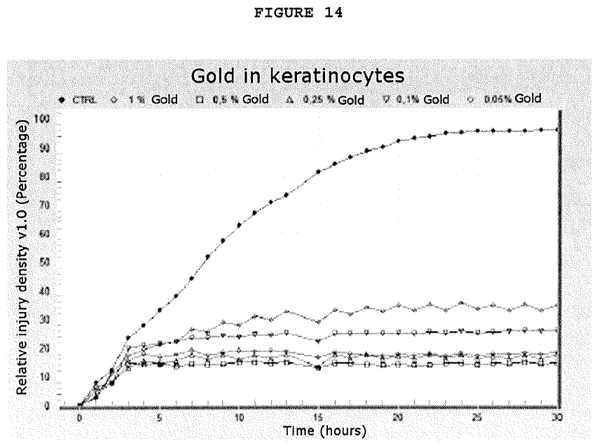

In relation to the method for repair and recovery of cutaneous tissue, the evaluation of the cellular migration effect is demonstrated by the speed with which the cells repair the artificial discontinuity of the created layer (wound maker) to demonstrate the effect that the revitalizing cosmetic compositions, specifically the Gold Colloidal Elixir and Diamond Elixir, present in potentiating the healing and recolonization of injury areas (FIGS. 11 to 12).

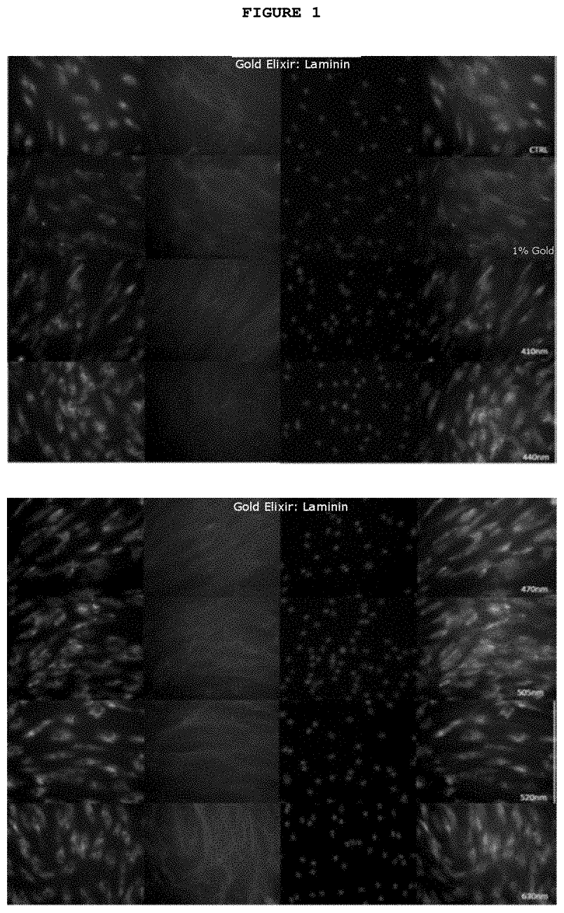

FIG. 1 shows qualitative microscopic images of increased laminin expression as a result of the synergistic potentiation of 1% application of the Gold Colloidal Elixir and LED exposure. The comparison was performed with control or only with the use of said elixir. Wherein:
Green: Specific marker signaling.
Red: Linking of phalloidin that highlights the cell actin filaments.
Blue: Identification of the cell nucleus.

The qualitative evaluation of Table 5 shows, in a relative way, how the LED wavelengths at 470 and 520 nm in contact with the Gold Colloidal Elixir potentiate laminin production by the fibroblasts. Data that is reinforced by the quantitative evaluation of FIG. 9, specifically at 470 nm, where exposure to the LED is maintained for 30 seconds.

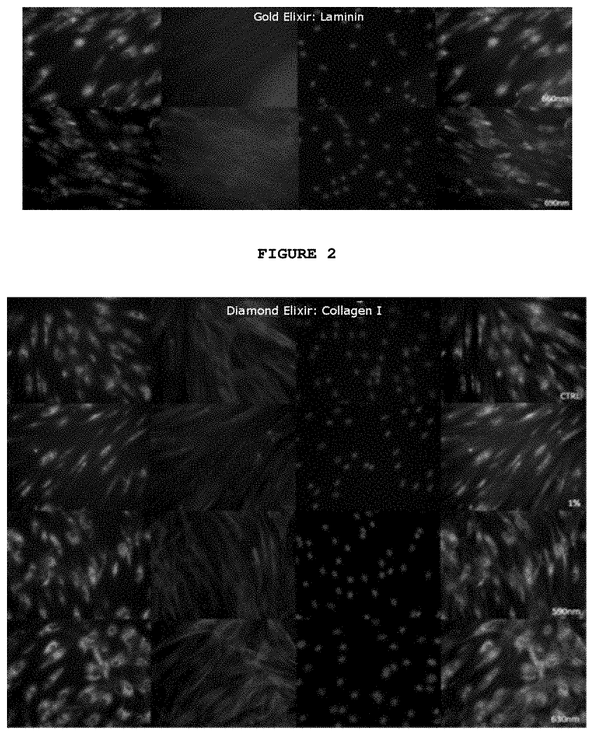

FIG. 2 shows qualitative microscopic captures of increased type I and III Collagen expression as a result of the synergistic potentiation of 1% application of the Diamond Elixir and LED exposure. The comparison was performed with control or only with the use of said elixir. Wherein:
Green: Specific marker signaling.
Red: Linking of phalloidin that highlights the cell actin filaments.
Blue: Identification of the cell nucleus.

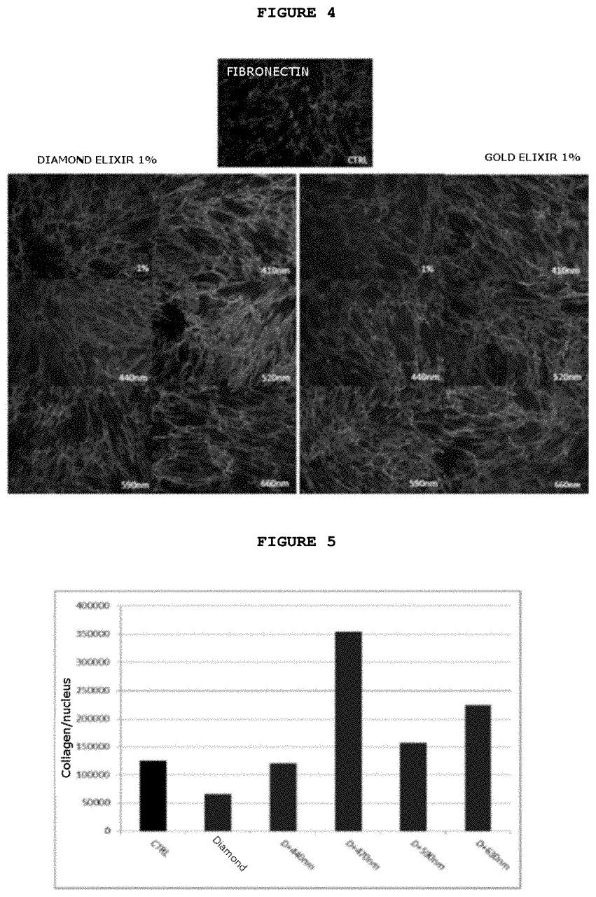

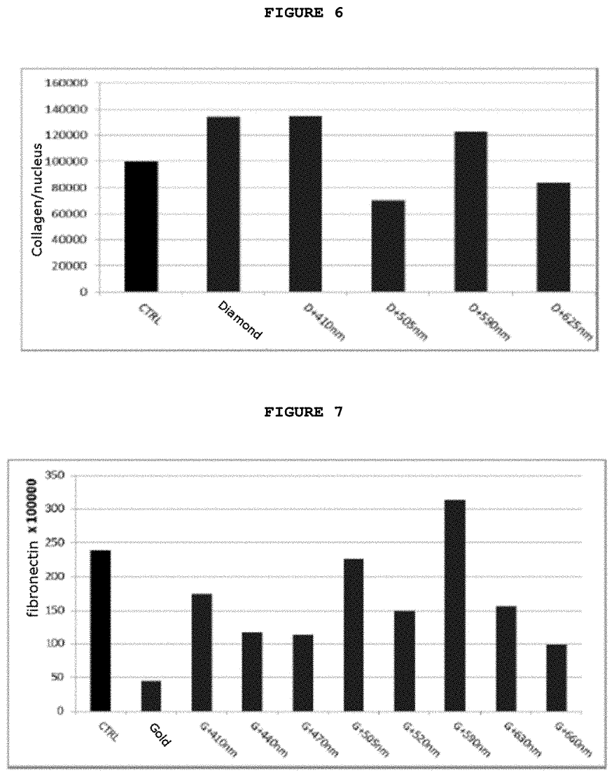

The qualitative evaluation of Tables 1 and 2 shows, in a relative way, how the LED wavelengths at 590, 630 and 660 nm in contact with the Diamond Elixir potentiate type I and III Collagen production by the fibroblasts. Data that are reinforced by the quantitative evaluation of FIGS. 5 and 6, specifically at 470 nm where the LED exposure is maintained for 30 seconds for type I collagen production and 590 nm where the exposure to the LED is maintained for 15 seconds for production of type III collagen.

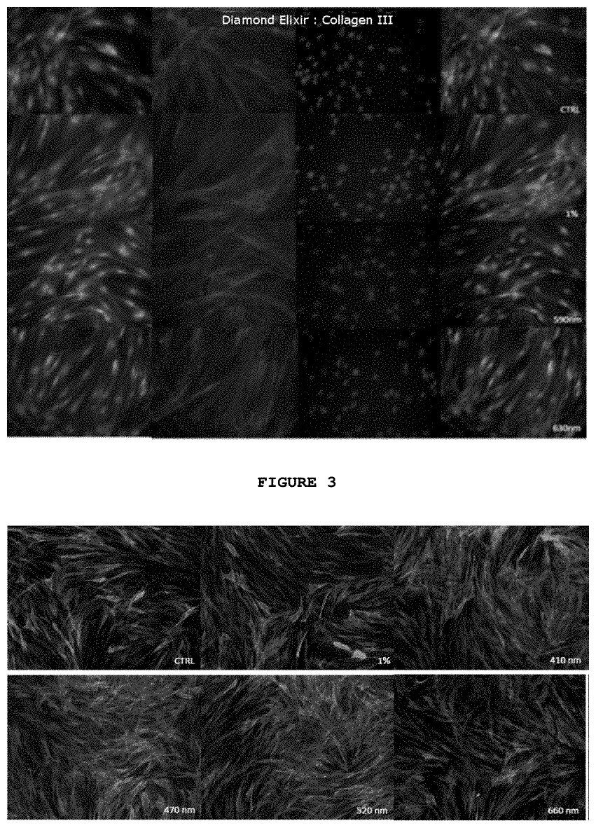

FIG. 3 shows qualitative microscopic images of increased α-SMA expression as a result of the synergistic potentiation of 1% application of the Diamond Elixir and LED exposure. The comparison was performed with control or only with the use of said elixir. Wherein:
Green: Specific marker signaling.

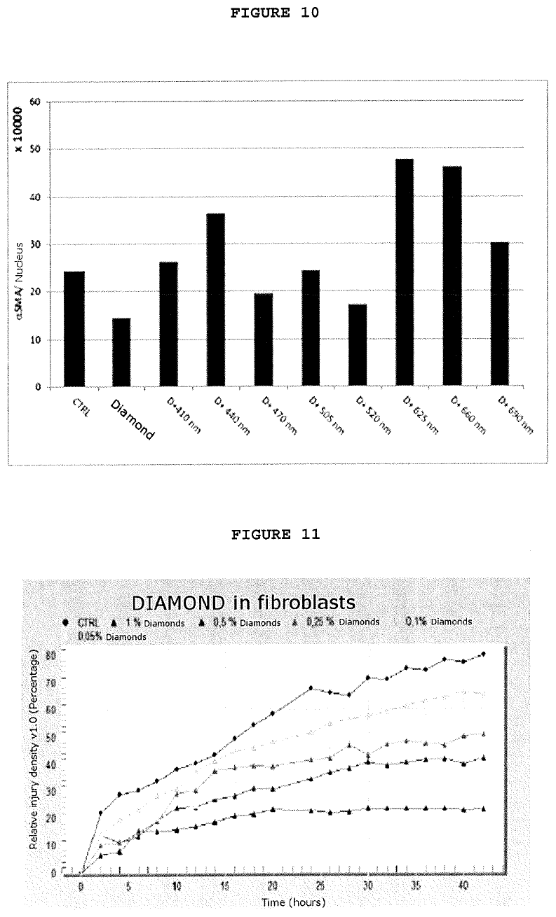

The qualitative evaluation of Table 4 shows, in a relative way, how the LED wavelengths at 590 nm in contact with the Diamond Elixir potentiate the manifestation of α-SMA marker by the fibroblasts. Data that is reinforced by the quantitative evaluation of FIG. 10, specifically at 625 and 660 nm, where exposure to the LED is maintained for 30 seconds.

FIG. 4 shows qualitative microscopic images of increased fibronectin expression as a result of the synergistic potentiation of 1% application of the Gold Elixir or Diamond Elixir and LED exposure. The comparison was performed with control or only with the use of said elixir. Wherein:
Green: Specific marker signaling.

The qualitative evaluation of Table 3 shows, in a relative way, how the LED wavelengths at 470, 520, 590 and 630 nm in contact with the Gold Colloidal Elixir potentiate fibronectin production by the fibroblasts. Data that is reinforced by the quantitative evaluation of FIG. 7, specifically at 590 nm, where exposure to the LED is maintained for 30 seconds.

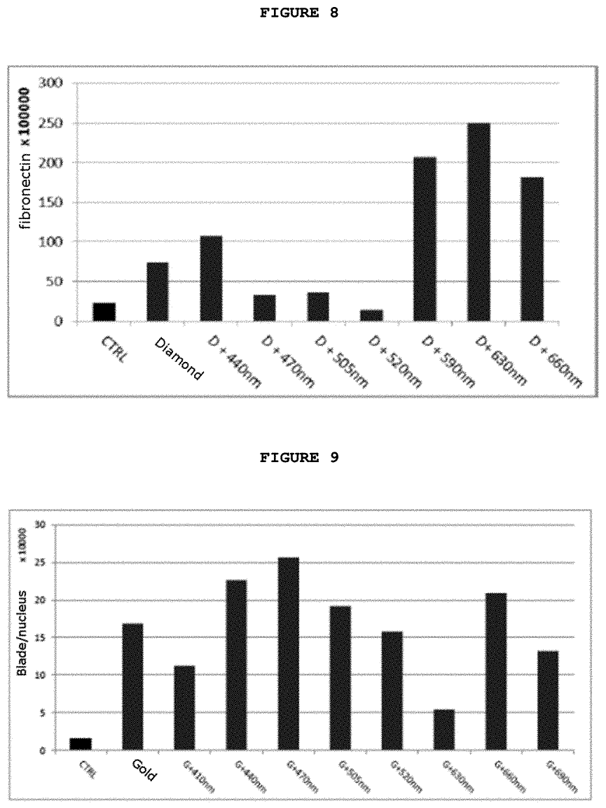

The qualitative evaluation of Table 3 shows, in a relative way, how the LED wavelengths at 440, 470, 520, 590, 630 and 660 nm in contact with the Diamond Elixir potentiate fibronectin production by the fibroblasts. Data that is reinforced by the quantitative evaluation of FIG. 8, specifically at 630 and 590 nm, where exposure to the LED is maintained for 30 seconds.

### Non-Therapeutic Method for Cutaneous Tissue Repair:

The fibroblast cell culture was submitted to LED and contact with the elixirs according to the best parameters of the qualitative evaluation (data from tables 1 to 5).

The evaluation of the cellular migration effect is demonstrated by the speed with which the cells repair the artificial discontinuity of the created layer (wound maker) to demonstrate the effect that the revitalizing cosmetic compositions, specifically the Gold Colloidal Elixir and Diamond Elixir, present in potentiating the healing and recolonization of injury areas.

Thus, FIGS. 11 and 12 depict the migration tests plots performed for fibroblasts.

FIG. 11 shows the results of fibroblast cell migration on different concentrations of the Diamond Elixir, identifying that the concentrations evaluated between 0.05% and 0.1% presented stimulus close to the control stimulated only by exposure to the LED.

FIG. 12 shows the results of fibroblast cellular migration on different concentrations of the Colloidal Gold Elixir, indicating that the concentrations evaluated between 1% and 0.5% presented a stimulus higher than the control stimulated only by exposure to the LED.

On a lower proportion and less potentiating effect when compared to the other results, but not less important, the association between LED exposure and Pearl & Caviar Elixir, increased the production of type I collagen in fibroblasts when exposed to 470, 590 and 690 nm. The manifestation of the α-SMA marker by fibroblasts was evidenced when exposed to 440, 470, 520, 630 and 660 nm. Fibronectin production in fibroblasts is higher when the Pearl & caviar Elixir is exposed at 630 and 690 nm.

On a lower proportion and less potentiating effect when compared to the other results, but not less important, the association between LED exposure and Platinum Elixir, increased the production of type I collagen in fibroblasts when exposed to 590 nm. The manifestation of the α-SMA marker by fibroblasts was evidenced when exposed to 590 and 780 nm. Fibronectin production in fibroblasts is higher when the Platinum Elixir is exposed at 660, 690 and 780 nm.

From the above description and example, it is possible to observe an increase in the synthesis of cellular proteins, or even in the proteins synthesized by the fibroblasts, proving an unexpected synergistic potentiating effect of the use of the revitalizing cosmetic compositions with LED exposure in relation to the state of the art.

Although certain embodiments have been specifically described, they have been presented only by way of example, and there is no intention to limit the scope of the invention. The claims accompanying this disclosure and its equivalents are considered to encompass such embodiments.

Finally, modifications of this invention apparent to a person skilled in the art, such as addition or removal of non-fundamental elements thereof, may be performed without departing from the scope and spirit of the invention.

1-2. (canceled)

3. A method for the skin aging treatment, comprising:
a. applying an acceptable amount of at least one revitalizing cosmetic composition onto the cutaneous tissue;
b. subjecting the revitalizing cosmetic composition applied in (a) to light having wavelengths between 410 nm and 780 nm;
c. maintaining the exposure of the revitalizing cosmetic composition to light as described in (b) for a time period ranging from at least 15 seconds to 2 minutes.

4. A method for repairing and recovering cutaneous tissue, comprising:
a. applying an acceptable amount of at least one revitalizing cosmetic composition onto injured or discontinued cutaneous tissue;
b. subjecting the revitalizing cosmetic composition applied in (a) to light having wavelengths between 410 nm and 780 nm;
c. maintaining the exposure of the revitalizing cosmetic composition as described in (b) for a time period ranging from 5 hours to 40 hours.

5. The method according to claim 3, wherein the skin markers are selected from the group consisting of: collagen I, collagen III, fibronectin, laminin and α-SMA.

6. The method according to claim 3, wherein the amount of the revitalizing cosmetic composition to be applied in cutaneous tissue varies between 0.35 and 15 ml.

7. The method according to claim 3, wherein the amount of the revitalizing cosmetic composition is still more preferably from 0.35 ml to 1.5 ml.

8. The method according to claim 4, wherein the amount of the revitalizing cosmetic composition to be applied in cutaneous tissue varies between 0.35 and 15 ml.

9. The method according to claim 4, wherein the amount of the revitalizing cosmetic composition is from 0.35 ml to 1.5 ml.

10. A method for the skin aging treatment, comprising:
a. applying an acceptable amount of at least one revitalizing cosmetic composition onto cutaneous tissue; and
b. subjecting the revitalizing cosmetic composition applied in (a) to light having wavelengths between 410 nm and 780 nm.
